(19) United States
(12) Patent Application Publication
(10) Pub. No.: US 2011/0131683 A1
(43) Pub. Date: Jun. 2, 2011

(54) Polypeptide Inducing Dwarfism of Plants, Polynucleotide Coding the Polypeptide, and Those Use

(75) Inventors: Dong-Hee Lee, Busan (KR); Kook-Jin Kim, Gyeongsangbuk-do (KR); Dong-Su Kim, Daegu (KR)

(73) Assignee: GENOMINE, INC., Pohang-si, Gyeongsangbuk-do (KR)

(21) Appl. No.: 12/597,167

(22) PCT Filed: Nov. 13, 2008
(86) PCT No.: PCT/KR2008/006679

§ 371 Date: May 21, 2010

(30) Foreign Application Priority Data
Nov. 13, 2007 (KR) 10-2007-0115458

(51) Int. Cl. A01H 1/06; A01H 5/00

(52) U.S. Cl. 800/290; 800/298

(57) ABSTRACT

Provided are polypeptides capable of inducing dwarfism in plants, polynucleotides encoding the same, and uses thereof.

### TECHNICAL FIELD

The present invention relates to a polypeptide which induces dwarfism in plants, a polynucleotide encoding the same, and the uses thereof. More particularly, the present invention relates to a polypeptide with GA 2-oxidase function responsible for the catabolism of gibberellin, a polynucleotide encoding the same, and uses thereof.

### BACKGROUND ART

Gibberellins (GAs) are tetracyclic diterpenoid phytohormones found in hundreds of various forms in plants. Of these forms, only several forms, such as GA₁, GA₃, GA₄, and GA₇, have bioactive functions. Such bioactive gibberellins are involved in the growth regulation and various developmental processes of plants, including germination, stem elongation, flowering, and leaf and fruit senescence.

Biologically, all known gibberellins are diterpenoid acids that are synthesized from the C₂₀ precursor GGDP (geranylgeranyl diphosphate) largely in the following three stages.

First, ent-kaurene is produced from GGDP through cyclization with catalysis by ent-copalyl diphosphate synthase (CPS) and ent-kaurene synthase (KS). In consideration of the cytosolic location of CPS and KS, the cyclization is inferred to occur in plastids (Sun and Kamiya, 1994; Helliwell et al., 2001).

In the second stage of gibberellin biosynthesis, ent-kaurene is oxidized to GA₁₂ by cytochrome P450 monoxygenase (P450s). This oxidation occurs on the plastid envelope and the endoplasmic reticulum (Helliwell et al., 2001).

In the final stage of gibberellin biosynthesis, GA₁₂ is converted to bioactive GA₄, which may be subdivided into two pathways catalyzed respectively by two 2-oxoglutarate dependent dioxygenases (2ODDs): conversion from GA₁₂ to GA₉ by GA 20-oxidase and from GA₉ to GA₄ by GA 3-oxidase. Interestingly, this final stage of gibberellin biosynthesis includes the catabolism of gibberellin, that is, the inactivation of gibberellin, by GA 2-oxidase, another form of 2ODDs, as well as the synthesis of active gibberellin by GA 20-oxidase and GA 3-oxidase. Recent studies have shown that the GA 2-oxidase of Arabidopsis may be further sub-classified to a group using C₂₀-Gas and intermediates rather than active gibberellins, as substrates (Thomas et al., 1999; Schomburg et al., 2003).

It has been reported that plant dwarfism is attributed to a deficiency in the quantity or signaling of some gibberellins (Peng et al., 1999; Spielmeyer et al., 2002). Accordingly, the inhibition or activation of enzymes involved in the gibberellin biosynthesis or degradation may induce plant dwarfism.

It is very important in crop breeding to induce plant dwarfism. Dwarfed crops show increased resistance to external stresses such as wind, rainfall, etc., bringing about an increase in crop harvest.

For this reason, those in the bioengineering field have made a great effort to find a polypeptide that is essentially responsible for inducing dwarfism in plants, or a polynucleotide encoding the same.

Under this background, the present invention has devolved.

### DISCLOSURE

### Technical Problem

It is therefore an object of the present invention to provide a polypeptide having a function of inducing dwarfism in plants.

It is an object of the present invention to provide a polynucleotide encoding the polynucleotide.

It is another object of the present invention to provide a method of preparing a dwarfed plant.

It is a further object of the present invention to provide a method of selecting a transgenic plant with dwarfism.

It is still a further object of the present invention to provide a method of providing such a dwarfed plant.

It is still another object of the present invention to provide a method of screening a plant dwarfism inducer.

### Technical Solution

As will be explained in greater detail, an Arabidopsis variety transformed with a GA 2-oxidase gene is found to have dwarfism induced in stems and leaves, but to be not different from the wild-type in root development and flowering time. The GA 2-oxidase gene was obtained by constructing a sense nucleotide from the full-length cDNA (SEQ ID NO. 1) prepared by PCR with the primers based on the base sequence of a GA 2-oxidase protein (GenBank accession number NP 175233) responsible for gibberellin catabolism in Arabidopsis.

It is also observed that the dwarfism-induced variety can be recovered to a phenotype of the wild-type by treatment with GA₃, a bioactive gibberellin.

Based on these experiments, the present invention is provided.

In accordance with an aspect thereof, the present invention provides a polypeptide capable of inducing dwarfism in plants.

The polypeptide capable of inducing plant dwarfism in accordance with the present invention is selected from among the following polypeptides (a), (b) and (c):

(a) a polypeptide having the entire amino acid sequence of SEQ. ID. NO. 2;

(b) a polypeptide containing a substantial part of the amino acid sequence of SEQ. ID. NO. 2; and

(c) a polypeptide substantially similar to that of (a) or (b).

As used herein, the phrase or term “a polypeptide containing a substantial part of the amino acid sequence of SEQ. ID. NO. 2” is defined as a polypeptide containing part of the amino acid sequence of SEQ. ID. NO. 2, which is long enough to still have the same function, essential for inducing dwarfism in plants, as the polypeptide consisting of the amino acid sequence of SEQ. ID. NO. 2. Any polypeptide, as long as it retains the essential function of inducing dwarfism in plants, satisfies the requirements of the present invention, and thus its length or activity is not important. That is, even if it is lower in activity than the intact polypeptide of SEQ. ID. NO. 2, any polypeptide that has the essential function for the induction of plant dwarfism may be included within the range of “the polypeptide that contains a substantial part of the amino acid sequence of SEQ. ID. NO. 2”, irrespective of the sequence length thereof. Those who are skilled in the art, that is, those who understand the prior art related to the present invention, expect that a deletion or an addition mutant of a polypeptide containing the amino acid sequence of SEQ. ID. NO. 2 will still retain the function of inducting plant dwarfism. As such, a polypeptide that contains the amino acid sequence of SEQ. ID. NO. 2, but from which an N- or C-terminal region has been deleted, is still functional. Generally, it is accepted in the art that even if its N-terminal region or C-terminal region is deleted therefrom, a mutant polypeptide can still retain the function of the intact polypeptide. As a matter of course, if the deleted N- or C-terminal region corresponds to a motif essential for the function of the peptide, the deleted polypeptide loses the function of the intact polypeptide. Nonetheless, the discrimination of such inactive polypeptides from active polypeptides is well known to those skilled in the art. Further, a mutant polypeptide which lacks a portion other than an N- or C-terminal region can still retain the function of the intact polypeptide. Also, those skilled in the art can readily examine whether or not such a deletion mutant still retains the function of the intact polypeptide. Particularly, in light of the fact that the present invention discloses the nucleotide sequence of SEQ. ID. NO. 1 and the amino acid sequence of SEQ. ID. NO. 2 and provides examples in which whether the polypeptide consisting of the amino acid sequence of SEQ. ID. NO. 2, encoded by the nucleotide sequence of SEQ. ID. NO. 1, has a plant dwarfism-inducing function was clearly examined, it will be clearly apparent that those who are skilled in the art can examine whether a deletion mutant of the polypeptide comprising the amino acid sequence of SEQ. ID. NO. 2 still functions like the intact polypeptide. Accordingly, it must be understood in the present invention that “a polypeptide containing a substantial part of the amino acid sequence of SEQ. ID. NO. 2” means any deletion mutant that can be prepared on the basis of the disclosure of the invention by those skilled in the art and that retains the plant dwarfism-inducing function.

As used in the foregoing and the following descriptions, including the claims, the phrase “a polypeptide substantially similar to that of (a) or (b)”, means a mutant that has at least one substituted amino acid residue but still retains the function of the amino acid sequence of SEQ. ID. NO. 2, that is, the plant dwarfism-inducing function. Likewise, if a mutant in which at least one amino acid residue is substituted still shows the plant dwarfism-inducing function, its activity or substitution percentage is not important. Accordingly, no matter how much lower a mutant polypeptide is in activity than a polypeptide containing the intact amino acid sequence of SEQ. ID. NO. 2, or no matter how much a mutant polypeptide has been substituted with amino acid residues compared to a polypeptide containing the intact amino acid sequence of SEQ. ID. NO. 2, the mutant polypeptide is included within the scope of the present invention as long as it shows the plant dwarfism-inducing function. Even if it has one or more amino acid residues substituted for a corresponding residue of the intact polypeptide, the mutant polypeptide still retains the function of the intact polypeptide if the substituted amino acid residue is chemically equivalent to the corresponding one. For instance, when alanine, a hydrophobic amino acid, is substituted with a similarly hydrophobic amino acid, e.g., glycine, or with a more hydrophobic amino acid, e.g, valine, leucine or isoleucine, the polypeptide(s) containing such substituted amino acid residue(s) still retain(s) the function of the intact polypeptide, even if it(they) has(have) lower activity. Likewise, a polypeptide(s) containing substituted amino acid residue(s), resulting from substitution between negatively charged amino acids, e.g., glutamate and aspartate, still retains the function of the intact polypeptide, even if it has lower activity. Also, this is true of a mutant polypeptide in which substitution occurs between positively charged amino acids. For example, a substitution mutant polypeptide, containing lysine instead of arginine, still shows the function of the intact polypeptide even if its activity is lower. In addition, polypeptides which contain substituted amino acid(s) in their N- or C-terminal regions still retain the function of the intact polypeptide. It is plainly obvious to those skilled in the art that current technology makes it possible to prepare a mutant polypeptide that retains the plant dwarfism-inducing function of the polypeptide containing the amino acid sequence of SEQ. ID. NO. 2, with at least one amino acid residue substituted therein. Also, those skilled in the art can examine whether a substitution mutant polypeptide still retains the function of the intact polypeptide. Further, because the present invention discloses the nucleotide sequence of SEQ. ID. NO. 1 and the amino acid sequence of SEQ. ID. NO. 2 and provides examples in which whether the polypeptide consisting of the amino acid sequence of SEQ. ID. NO. 2, encoded by the nucleotide sequence of SEQ. ID. NO. 1, has a plant dwarfism inducing function was clearly examined, it will be very apparent that “the polypeptide substantially similar to that of (a) or (b)” can be readily prepared by those who are skilled in the art. Accordingly, the “polypeptide substantially similar to that of (a) or (b)” is understood to include all polypeptides that have the plant dwarfism-inducing function, in spite of the presence of one or more substituted amino acids therein.

Although “a polypeptide substantially similar to that of (a) or (b)” means any mutant that has at least one substituted amino acid residue but still retains the plant dwarfism-inducing function, a polypeptide which shares higher homology with the amino acid sequence of SEQ. ID. NO. 2 is more preferable from the point of view of activity. Useful is a polypeptide that shows 60% or higher homology with the wild-type polypeptide, with the best preference for 100% homology. In more detail, more preferable are sequence homologies of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99%, in ascending order of preference.

Because “the polypeptide substantially similar to that of (a) or (b)” includes polypeptides substantially similar to “the polypeptide containing a substantial part of the amino acid sequence of SEQ. ID. NO. 2” as well as polypeptides substantially similar to “the polypeptide having an amino acid sequence 100% coincident with SEQ. ID. NO. 2”, the above description is true both for polypeptides substantially similar to “the polypeptide having the entire amino acid sequence of SEQ. ID. NO. 2” and for polypeptides substantially similar to “the polypeptide containing a substantial part of the amino acid sequence of SEQ. ID. NO. 2”.

In accordance with another aspect thereof, the present invention provides an isolated polynucleotide encoding the above-mentioned polypeptide.

Herein, the term “the above-mentioned polypeptide” is intended to include not only the polypeptide having the amino acid sequence of SEQ. ID. NO. 2, polypeptides containing a substantial part of the amino acid sequence of SEQ. ID. NO. 2, and polypeptides substantially similar to these peptides, but also all polypeptides that retain the plant dwarfism-inducing function in the preferred embodiments. If an amino acid sequence is revealed, a polynucleotide encoding the amino acid sequence can be readily prepared on the basis of the amino acid sequence by those skilled in the art.

In the present invention, the phrase “the isolated polynucleotide”, as used herein, is intended to include all chemically synthetic polynucleotides, isolated polynucleotides from living bodies, especially Arabidopsis thaliana, and polynucleotides containing modified nucleotides, whether single- or double-stranded RNA or DNA. Accordingly, cDNAs, chemically synthetic polynucleotides, and gDNAs isolated from living bodies, especially Arabidopsis thaliana, fall into the range of “the isolated polynucleotide”. On the basis of the amino acid sequence of SEQ. ID. NO. 2, and the nucleotide sequence of SEQ. ID. NO. 1, encoding the amino acid sequence therefor, and technology known in the art, the preparation of corresponding cDNAs and chemically synthetic polynucleotides and the isolation of gDNA can be readily achieved by those who are skilled in the art.

In accordance with a further aspect thereof, the present invention provides a method for preparing a dwarfed plant.

The method may be carried out in two manners.

In a first embodiment, a dwarfed plant can be prepared by (I) transforming the above-mentioned polynucleotide encoding a polypeptide capable of inducing dwarfism in plants into a plant and (II) selecting a dwarfism-induced plant from among the resulting transformants.

As is apparent in the following examples, the Arabidopsis thaliana mutant with the base sequence of SEQ ID NO. 1 introduced thereinto is found to show dwarfism in the stems and leaves thereof.

The term “dwarfism”, as used herein, is used to mean that the biomass of a plant is less than that of the wild-type, preferably in the stems and/or leaves. Herein, biomass may be understood to indicate weight, length and/or size of plant organs, such as leaves, stems, etc.

Also, the “polynucleotide” of the present invention is intended to include all polynucleotides which encode the polypeptides capable of inducing dwarfism in plants. For this reason, the polynucleotide must be understood to include all of the polynucleotides mentioned in the preferred embodiments. Nonetheless, the polynucleotide is preferably a polynucleotide coding for the amino acid sequence of SEQ ID NO. 2 and more preferably a polynucleotide containing the base sequence of SEQ ID NO. 1.

As used herein, the term “plant” is intended to include all plants which produce results beneficial to humans when their biomass is decreased. The most direct examples of such plants include various weeds inhibitory of the growth of crops, potted plants, flowering plants, etc. In addition, edible plants may fall into the range of being considered plants on the grounds of resistance to external stress (wind, rainfall), the simplicity of eating them, convenience of their transportation, etc. In greater detail, the examples of the plant include weeds growing on arable lands, potted plants such as roses, pine trees, nut pines, bamboos, etc., flowering plants such as gladiola, gerberas, carnations, chrysanthemums, lilies, tulips, etc., edible plants such as rice, wheat, barley, corn, bean, potato, red bean, oats, millet, Chinese cabbage, radish, pepper, strawberry, tomato, water melon, cucumber, cabbage, melon, pumpkin, Welsh onion, onion, carrot, ginseng, tobacco, cotton, sesame, sugarcane, sugar beet, perilla, peanut, canola, apple tree, pear tree, jujube tree, peach, kiwi, grape, tangerine, persimmon, plum, apricot, banana, etc., and fodder plants such as rye grass, red clover, orchard grass, alphalpha, tall fescue, perennial rye grass, etc., but are not limited thereto.

Also, the term “plant”, as used herein, must be understood to include not only adult plants, but also plant cells, tissues, and seeds which can develop into adult plants.

As used herein, the term “transformation” is intended to mean the genotypic alteration of a host plant resulting from the introduction of an exogenous polynucleotide (i.e., a polynucleotide coding for a dwarfism-inducing polypeptide). That is, transformation refers to the introduction of a foreign genetic material into a host plant, more accurately, a host plant cell, irrespective of the method used therefor. When introduced into a host cell, the exogenous polypeptide may be integrated into the genome or remain in the cytosol, and both of these possibilities are included within the scope of the present invention.

The techniques of transforming plants with exogenous polynucleotides are well known in the art (Methods of Enzymology, Vol. 153, 1987, Wu and Grossman Ed., Academic Press).

For the transformation of plants, a vector, such as a plasmid or virus, anchoring the exogenous polynucleotide thereto, or a mediator such as Agrobacterium spp. (Chilton et al., 1977, Cell 11:263:271) may be used. Also, an exogenous polynucleotide may be directly introduced into plant cells (Lorz et al., 1985, Mol. Genet. 199:178-182).

Widely used is a plant transformation method in which Agrobacterium tumefaciens harboring an exogenous polynucleotide is transfected into young plants, plant cells or seeds. Those skilled in the art can culture and grow the transfected plant cells or seeds into mature organisms.

The transforming step (I) is preferably carried out by (a) inserting a polynucleotide encoding a plant dwarfism-inducing polypeptide in an operably linking manner into an expression vector containing a regulatory nucleotide sequence to construct a recombinant expression vector and (b) introducing the recombinant vector into a host plant to afford a transgenic plant.

Preferably, the transforming step (1) comprises inserting a polypeptide encoding a plant dwarfism-inducing polypeptide in an operably linking manner into an expression vector containing a regulatory nucleotide sequence to construct a recombinant expression vector, transforming an Agrobacterium spp. with the recombinant expression vector, and transfecting the transformed Agrobacterium spp. into a plant. More preferably, the transformed Agrobacterium spp. is transformed Agrobacterium tumefaciens.

The term “regulatory nucleotide sequence” must be understood to include all sequences that have influence on the expression of the gene of interest. Examples of the regulatory nucleotide sequence include leader sequences, enhancers, promoters, transcription initiation region, transcription termination region, replication origin, etc.

The term “operably linking” or “operably linked”, as used herein, is used to mean that a regulatory sequence is functionally linked to another nucleotide sequence, thereby regulating the transcription and/or translation of this nucleotide sequence.

As for promoter sequences useful in the present invention, they may be inducible or constitutive. Representative of constitutive promoters are CaMV promoters and Nos promoters. Examples of inducible promoters (the activity of the promoter is induced by an inducer to express an operably linked gene) include a yeast-copper metallothionein promoter (Mett et al., Proc. Natl. Acad. Sci., U.S.A., 90:4567, 1993), substituted benzenesulfonamide-inducible In2-1 and In2-2 promoters (Hershey et al., Plant Mol. Biol., 17:679, 1991), a glucocorticoid response element (GRE) (Schena et al., Proc. Natl. Acad. Sci., U.S.A., 88:10421, 1991), an ethanol-inducible promoter (Caddick et al., Nature Biotech., 16:177, 1998), a light-inducible promoter from the small subunit of ribulose-1,5-bisphosphate carboxylase (ssRUBISCO) (Coruzzi et al., EMBO J., 3:1671, 1984; Broglie et al., Science, 224:838, 1984), a manopine synthase promoter (Velten et al., EMBO J., 3:2723, 1984), nopaline synthase (NOS) and octopine synthase (OCS) promoters, a heat-shock promoter (Gurley et al., Mol. Cell. Biol., 6:559, 1986; Severin et al., Plant Mol. Biol., 15:827, 1990).

The recombinant vector may harbor a selectable marker gene. The term “marker gene”, as used herein, is intended to refer to a gene encoding a character which allows the selection of the plant or plant cell containing the gene. Marker genes may be resistant to antibiotics or herbicides. Examples of the selectable marker genes useful in the present invention include an adenosine deaminase gene, a dihydrofolate reductase gene, hydromycin-B-phosphotransferase gene, a thymidine kinase gene, a xanthine-guanine phosphoribosyl transferase, and a phosphinotricine acetyltransferase gene.

In an embodiment of the present invention, a gene consisting of the base sequence of SEQ ID NO. 1 is inserted into the expression vector pSEN to construct a recombinant vector pSEN-AtGA2ox4 which is in turn transformed into Agrobacterium tumefaciens, followed by the transfection of the transformed Agrobacterium tumefaciens into Arabidopsis thaliana.

When the embodiment of the present invention is taken into consideration, the step (I) preferably comprises transforming a plant with a gene consisting of the base sequence of SEQ ID NO. 1 and more preferably with a recombinant vector containing the gene, especially pSEN-AtGA2ox4, and most preferably transfecting Agrobacterium tumefaciens carrying the vector, especially pSEN-AtGA2ox4, into a plant.

The selecting step (II) may be carried out by selecting plants with the naked eye after the growth of the transgenic or transformed plant of step (I) or by taking advantage of a selectable marker gene introduced at the same time into the plant.

In a second embodiment of the present invention, a dwarfed plant can be prepared by (I) overexpressing a gene consisting of the base sequence of SEQ ID NO. 1 or a gene consisting of a base sequence similar to that of SEQ ID NO. 1, and (II) selecting a dwarfism phenotype-induced plant.

As used herein, the phrase “a gene consisting of a base sequence similar to that of SEQ. ID. NO. 1” is intended to include all genes that are homologs of the gene of SEQ. ID. NO. 1, with the retention of the plant dwarfism-inducing function, and yet are different in nucleotide sequence from the base sequence of SEQ. ID. NO. 1 due to evolutionary differences between plants. More preferable from the point of view of activity is a gene consisting of a base sequence similar to that of SEQ. ID. NO. 1, which shares higher homology with the base sequence of SEQ. ID. NO. 1. Useful is a gene that shows 60% or higher homology with the wild-type gene, with the best preference for 100% homology. In more detail, more preferable are sequence homologies of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99%, in ascending order of preference.

The term “overexpression”, as used herein, refers to an expression which exceeds the normal expression in the wild-type plant.

The overexpression of a gene consisting of a base sequence identical or similar to that of SEQ ID NO. 1 may be accomplished chemically or by genetic engineering as explained in the first embodiment. In the method of preparing a dwarfed plant according to the present invention, therefore, the step (I) may be conducted by overexpressing a gene consisting of a base sequence identical or similar to that of SEQ ID NO. 1 with the aid of a chemical or by using genetic engineering.

The selection step (II) may be carried out with the naked eye or by taking advantage of a selectable marker if it is introduced into the plant.

In accordance with a still further aspect thereof, the present invention provides a dwarfed plant prepared using the method.

In accordance with still another aspect thereof, the present invention provides a method of preparing a plant with an improvement in seed productivity.

In an embodiment of this aspect, the method of preparing a plant with an improvement in seed productivity comprises (I) transforming a plant with the above-mentioned polynucleotide encoding a plant dwarfism-inducing polypeptide, and (II) selecting a dwarfism-induced plant.

In another embodiment of this aspect, the method of preparing a plant with an improvement in seed productivity comprises (I) overexpressing a gene consisting of a base sequence identical or similar to that of SEQ ID NO. 1 and (II) selecting a dwarfism-induced plant.

As will be elucidated in more detail in the following examples, the overexpression of the gene of SEQ ID NO. 1 encoding a plant dwarfism-inducing polypeptide in Arabisopsis thaliana leads to inducing dwarfism in the plant, resulting in a significant increase in seed productivity as compared to the wild-type. The term “seed productivity” means the number of seeds produced by one plant. Also, the term “plant with an improvement in seed productivity” indicates plants which are of higher seed productivity than is the wild-type.

The description of the method of preparing a dwarfed plant is applicable to the steps (I) and (II) of both the above embodiments,

In accordance with a still further aspect thereof, the present invention provides a plant with an improvement in seed productivity prepared by the method.

In accordance with yet another aspect thereof, the present invention provides a method of selecting a transgenic plant using the above-mentioned polynucleotide of the present invention as a marker gene.

The method of selecting a transgenic plant in accordance with the present invention comprises (I) transforming a plant with an expression vector carrying a target gene, an above-mentioned polynucleotide encoding a plant dwarfism-inducing polypeptide, and a regulatory nucleotide sequence, and (II) discriminating a dwarfism-induced plant variety from the non-induced one.

As used herein, the term “target gene” is defined as a polynucleotide sequence encoding a product of interest, be it natural or mutant (i.e., RNA or polypeptide). The target gene may be cDNA or gDNA in an isolated, fused or tagged form.

The step (I) of transforming a plant with an expression vector may be carried out by transforming the expression vector into Agrobacterium spp. and transfecting the transformed Agrobacterium spp. into the plant. The Agrobacterium spp. is preferably Agrobacterium tumefaciens.

As elucidated in the following examples, if necessary, the phenotype of the dwarfism-induced plant may be recovered back to that of the wild-type by treatment with GA₃.

For the method of selecting a transgenic plant, the description of the method for preparing a dwarfed plant in accordance with the present invention is applicable.

In accordance with yet still another aspect thereof, the present invention provides a method of screening a plant dwarfism inducer.

This method comprises (I) treating a plant with a chemical or biological material, and (II) detecting the inducer which causes the expression of a gene consisting of a base sequence identical or similar to that of SEQ ID NO. 1.

The term “gene consisting of a base sequence similar to that of SEQ ID NO. 1” may refer to the description of the method of preparing a dwarfed plant according to the present invention.

The treating step (I) may be conducted by bringing the plant into contact with a chemical or by using a bioengineering technique as described when describing the method of preparing a dwarfed plant.

As candidates for the plant dwarfism inducer, examples include the sense nucleotide sequence of SEQ ID NO. 1, a recombinant vector carrying the sense nucleotide sequence, and Agrobacterium tumefaciens transformed with the recombinant vector.

### Advantageous Effects

As described in the above, the polypeptide having a function of inducing dwarfism in plants, and a polynucleotide encoding the polypeptide are provided.

Also, a method is provided for preparing a dwarfed plant. The dwarfed plant thus prepared is provided. In addition, a method for screening a plant dwarfism inducer is provided.

### DESCRIPTION OF DRAWINGS

FIG. 1 is a schematic view showing the structure of a pSEN vector into which a plant dwarfism-inducing gene composed of the base sequence of SEQ ID NO. 1 will be introduced in a sense or antisense direction.

FIG. 2 is a schematic view showing the structure of the pSEN-AtGA2ox4 recombinant vector constructed by inserting the plant dwarfism-inducing gene composed of the base sequence of SEQ ID NO. 1 in a sense direction to the pSEN vector of FIG. 1.

FIG. 3 is a schematic view showing the structure of the pSEN-antiAtGA2ox4 recombinant vector constructed by inserting the plant dwarfism-inducing gene composed of the base sequence of SEQ ID NO. 1 in an antisense direction to the pSEN vector of FIG. 1.

FIGS. 4 and 5 are photographs showing T₂ lines of the Arabidopsis thaliana transformed with the pSEN-AtGA2ox4 and the pSEN-antiAtGA2ox4 recombinant vector of FIGS. 2 and 3, grown for 30 and 48 days, respectively, after germination. In these drawings, Col-O stands for wild-type Arabidopsis thaliana, SEN::AtGA2ox4-10 for the tenth transformant of the T₂ line of the Arabidopsis thaliana transformed with the pSEN-AtGA2ox4 recombinant vector, and atga2ox4-4 for the fourth transformant of the T₂ line of the Arabidopsis thaliana transformed with pSEN-antiAtGA2ox4 recombinant vector.

FIG. 6 is a graph showing the numbers of leaves that the ninth and the tenth transformants of the T2 lines of the Arabidopsis thaliana transformed with the pSEN-AtGA2ox4 recombinant vector have at the time of flowering.

FIG. 7 is a graph showing seed productivity (seed numbers per plant) of the T2 lines of the Arabidopsis thaliana transformed with pSEN-AtGA2ox4 and pSEN-antiAtGA2ox4 recombinant vectors, in which Col-O, SEN::AtGA2ox4-10 and atga2ox4-4 stand for the same things as they do in FIGS. 4 and 5.

FIG. 8 shows an RT-PCR analysis for expression patterns of GA2 oxidase-related genes and flowering control-related genes including the AtGA2ox4 gene in various organs of the wild-type (Col-O) and the dwarfism-induced mutant SEN::AtGA2ox4, both grown for 30 days after germination.

FIG. 9 shows an RT-PCR analysis for expression patterns of gibberellin biosynthesis-related genes in various organs. In FIGS. 8 and 9, “F” stands for flowers, “R” for roots, “S” for stems, “L” for leaves, “Si” for siliques, and AtGA2ox1, AtGA2ox2, AtGA2ox3, AtGA2ox4, AtGA2ox6, AtGA2ox7 and AtGA2ox8 are GA 2-oxidase-related genes of Arabidopsis thaliana, FT and CO are flowering control-related genes, and AtGA20ox1, AtGA20ox2 and AtGA3ox1 are gibberellin biosynthesis-related genes.

FIG. 10 is a photograph showing Arabidopsis thaliana varieties grown for 30 days after germination from the seeds of the ninth (SEN::GA2ox4-9) and the tenth T₂ lines (SEN::GA2ox4-10) of Arabidopsis thaliana transformed with the pSEN-AtGA2ox4 recombinant vector, with GA3 applied thereto twice at regular intervals of one week starting from 12 days after germination.

FIG. 11 is a graph showing lengths of the Arabidopsis thaliana varieties, in which Col-O stands for the wild-type, and SEN::GA2ox4-9 and SEN::GA2ox4-10 are defined as in FIG. 10.

FIG. 12 is a photograph showing Arabidopsis thaliana varieties grown for 40 days after germination from the seeds of the ninth (SEN::GA2ox4-9) and the tenth T₂ lines (SEN::GA2ox4-10) of Arabidopsis thaliana transformed with the pSEN-AtGA2ox4 recombinant vector, with GA3 applied thereto twice at regular intervals of one week starting from 12 days after germination.

FIG. 13 is a two-dimensional electrophoresis analytical gel showing the expression pattern of proteins from the wild-type Arabidopsis thaliana grown for 30 days after germination. In this drawing, spots represented by numerals are proteins up-regulated by the overexpression of AtGA2ox4 and recovered to wild-type levels by treatment with GA₃.

FIG. 14 is a two-dimensional electrophoresis analytical gel showing the expression pattern of proteins from the dwarfism-induced Arabidopsis thaliana mutant SEN::GA2ox4 grown for 30 days after germination.

FIG. 15 is a two-dimensional electrophoresis analytical gel showing the expression pattern of proteins from the dwarfism-induced Arabidopsis thaliana mutant SEN::GA2ox4, grown for 30 days after germination, the phenotype of which was recovered to the wild-type by treatment with GA3 twice at regular intervals of one week starting from 12 days after germination.

### MODE FOR INVENTION

A better understanding of the present invention may be obtained through the following examples which are set forth to illustrate, but are not to be construed as limiting the present invention

### Example 1

### Isolation of a Gene Encoding a Polypeptide Having a Plant Dwarfism Inducing function from Arabidopsis thaliana

The following processes were performed for isolating a gene, encoding a polypeptide having a plant dwarfism inducing function, from Arabidopsis thaliana.

### Example 1-1

### Cultivation and Nurturance of Arabidopsis thaliana

Arabidopsis thaliana was cultured in soil in pots or in an MS medium (Murashige and Skoog salts, Sigma, USA) containing 2% sucrose (pH 5.7) and 0.8% agar in Petri dishes. When using pots, the plants were cultivated at 22° C. under a light-dark cycle of 16/8 hours in a growth chamber.

### Example 1-2

### RNA Isolation and cDNA Library Construction

In order to construct Arabidopsis thaliana cDNA libraries, first, total RNA was isolated from all organs of Arabidopsis thaliana in various stages of differentiation using an RNeasy Plant Mini kit (QIAGEN, Germany). From the isolated total RNA, cDNA was prepared with the aid of Superscript III Reverse Transcriptase (INVITROGEN, USA).

### Example 1-3

### Isolation of a Gene Encoding a Polypeptide Having a Plant Dwarfism Inducing Function

Based on the amino acid sequence of AtGA2ox4 (GeneBank accession number NP 175233), a member of the GA 2-oxidase family using C₁₉-GAs (gibberellins) as a substrate, of Arabidopsis thaliana, a sense primer, represented by SEQ ID NO. 3, containing a BamHI site, and an antisense primer, represented by SEQ ID NO. 4, containing a BstEII site, were synthesized. Using these two primers, a full-length cDNA was amplified through PCR (polymerase chain reaction) from the cDNA library constructed in Example 1-2.

The cDNA was analyzed to have a 966 by open reading frame (ORF) of SEQ ID NO. 1, composed of three exons, encoding a polypeptide consisting of 321 amino acid residues with a molecular weight of about 35.9 kDa, and was called AtGA2ox4 (Arabidopsis thaliana GA 2-oxidase 4) or AtGA2ox4 gene. Its protein is expressed as “AtGA2ox4” or “AtGA2ox4 protein”. The AtGA2ox4 protein encoded by the gene was found to have an isoelectric point of 6.72.

Because this protein was suggested to act as a GA 2-oxidase responsible for the catabolism of gibberellins, the polynucleotide of the present invention was analyzed for GA 2-oxidase activity using mutants of Arabidopsis thaliana.

### Example 2

### Preparation and Characterization of Arabidopsis Thaliana Mutant Harboring Sense AtGA2ox4 Gene and Antisense Construct Complementary to AtGA2ox4 Gene

### Example 2-1

### Preparation of Arabidopsis thaliana Mutant Harboring Sense AtGA2ox4 Gene and Antisense Construct Complementary to AtMSG Gene

In order to examine whether the gene is involved in the induction of dwarfism in plants, the AtGA2ox4 gene was introduced in the sense and antisense directions into Arabidopsis thaliana to alter the expression of the AtGA2ox4 transcript.

AtGA2ox4 cDNA was amplified from the cDNA library of Arabidopsis thaliana through PCR using a sense primer, represented by SEQ ID NO. 3, containing a BamHI site, and an antisense primer, represented by SEQ ID NO. 4, containing a BstEII site. The PCR product thus obtained was digested with restriction enzymes BamHI and BstEII and inserted in a sense direction into the pSEN vector, under the control of the inducible promoter sen1, to construct a recombinant vector, named pSEN-AtGA2ox4, harboring an AtGA2ox4 gene.

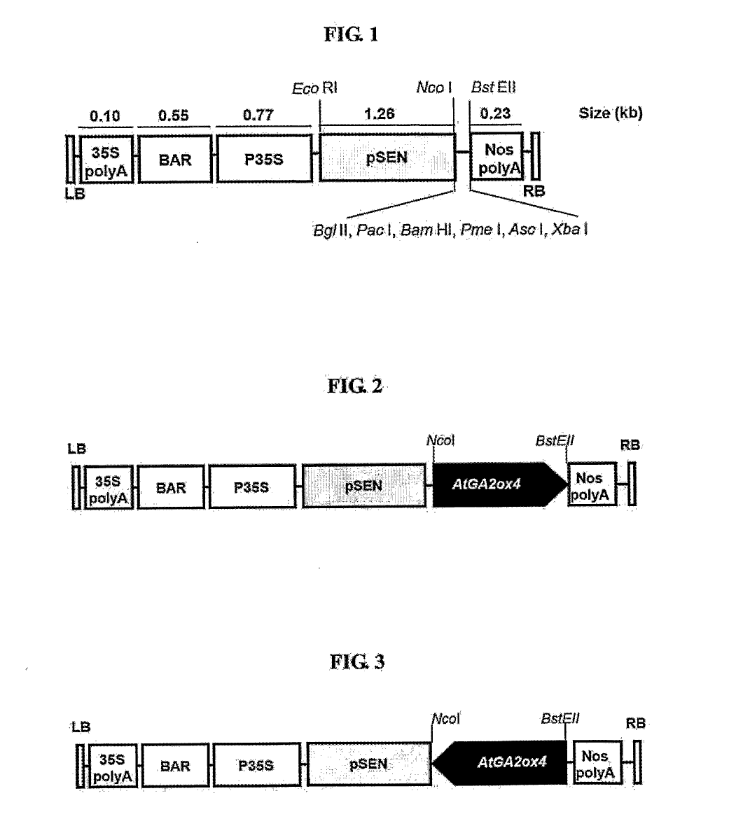

Likewise, AtGA2ox4 cDNA was amplified from the cDNA library of Arabidopsis thaliana through PCR using a sense primer, represented by SEQ ID NO. 5, containing a BstEII site, and an antisense primer, represented by SEQ ID NO. 6, containing a BamHI site. The PCR product thus obtained was digested with restriction enzymes BamHI and BstEII and inserted in a sense direction into the pSEN vector, under the control of the inducible promoter sen1, to construct a recombinant vector, named pSEN-antiAtGA2ox4, harboring an AtGA2ox4 gene. The sen1 promoter shows specificity for the genes expressed according to growth stages. FIGS. 1 to 3 respectively show the structures of the pSEN vector, the pSEN-AtGA2ox4 recombinant vector with the AtGA2ox4 gene introduced in the sense direction thereinto, and the pSEN-antiAtGA2ox4 recombinant vector with the AtGA2ox4 gene introduced thereinto in the antisense direction. In FIGS. 1 to 3, BAR stands for a bar gene (phosphinothricin acetyltransferase gene) conferring Basta resistance, RB for a right border, LB for a left border, P35S for a CaMV 35S RNA promoter, 35S poly A for CaMV 35S RNA poly A, PSEN for a sen1 promoter, and Nos polyA for nopaline synthase gene polyA.

The pSEN-AtGA2ox4 and the pSEN-antiAtGA2ox4 recombinant vector were separately introduced into Agrobacterium tumefaciens using an electroporation method. The transformed Agrobacterium strains were cultured at 28° C. to an O.D.₆₀₀ of 1.0, followed by harvesting cells by centrifugation at 25° C. at 5,000 rpm for 10 min. The cell pellets thus obtained were suspended in infiltration media (IM: 1×MS SALTS, 1X B5 vitamin, 5% sucrose, 0.005% Silwet L-77, Lehle Seed, USA) until O.D.₆₀₀ reached 2.0. Four week-old Arabidopsis thaliana was immersed in the Agrobacterium suspension in a vacuum chamber and allowed to stand for 10 min under a pressure of 10⁴ Pa. Thereafter, the Arabidopsis thaliana was placed for 24 hours in a polyethylene bag. The transformed Arabidopsis thaliana strains were grown to obtain seeds (T1). Arabidopsis thaliana, wild-type or transformed only with a vector (pSEN) carrying no AtGA2ox4 genes, was used as a control.

### Example 2-2

### Characterization of Transformed T1 and T2 Arabidopsis thaliana

After being immersed in a 0.1% Basta herbicide solution (Kyung Nong Co. Ltd., Korea) for 30 min, seeds from the Arabidopsis thaliana transformed in Example 2-1 were cultured. A Basta herbicide was applied five times to each pot in which the transformed Arabidopsis thaliana grew, and observation was made of the growth pattern of the Arabidopsis thaliana in each pot. Compared to the control (Arabidopsis thaliana transformed only with a vector (pSEN) carrying no AtGA2ox4 genes or wild-type Arabidopsis thaliana), the T₁ Arabidopsis thaliana transformed with the pSEN-AtGA2ox recombinant vector was surprisingly observed to have dwarfism induced in almost all the organs thereof. Various extents of dwarfism were believed to result from differences in gene overexpression from one individual to another. In contrast, no noticeable phenotype changes were induced in the T1 Arabidopsis thaliana transformed with the pSEN-antiAtGA2ox recombinant vector as compared to the control.

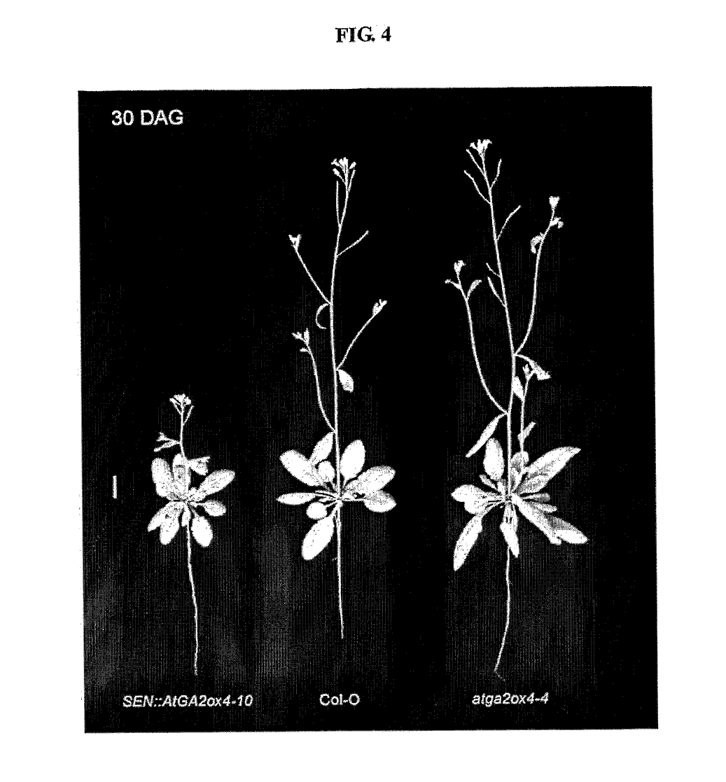

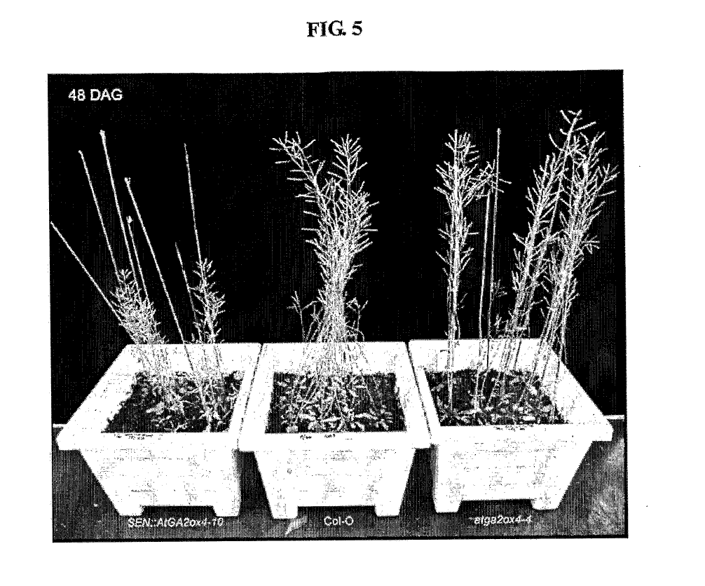

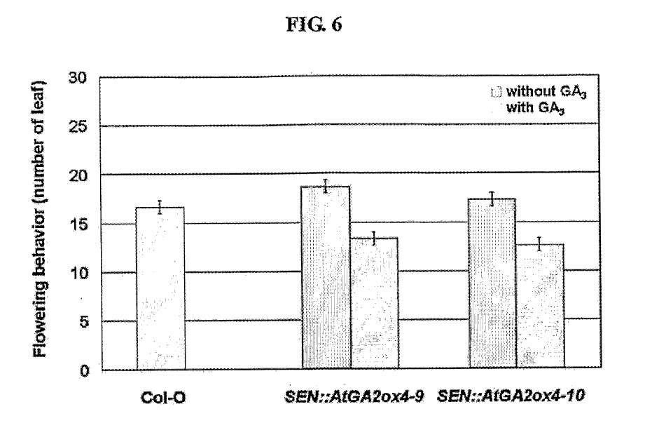

The phenotype of these transformed Arabidopsis thaliana mutants was examined. For this, T₂ seeds were obtained from the T₁ line of the transformed Arabidopsis thaliana. Thirty T₂ seeds, which had been subjected to low temperature treatment (4° C.) for 3 days, were cultured in pots and then treated with a Basta herbicide to select transformed plants. Phenotypes of the individual plants cultured for 30 days (FIG. 4) and 48 days (FIG. 5) after germination were examined. Like the T₁ mutant, the SEN::AtGA2ox4-10 mutant line with the pSEN-AtGA2ox4 construct was observed to have dwarfism induced in most organs including leaves, stems, etc., as compared to Col-O (wild-type). This dwarfism was different in extent from one individual to another, which was believed to result from differences in overexpression extent. However, there were no significant differences in root development and flowering time between the mutant and the wild-type (FIG. 6). It was inferred that the dwarfism induction might be attributed to an insufficient level of active gibberellins because they were converted to inactive forms due to the overexpression of AtGA2ox4, which uses C₁₉-GAs (gibberellins) as substrates. On the other hand, the atga2ox4-4 mutant line with the pSEN-antiAtGA2ox4 construct was slightly taller and thinner than the wild-type, with the stem extended longer. However, no significant phenotype differences were found between the atga2ox4-4 mutant line and the wild-type (FIGS. 4 and 5). The suppression of dwarfism phenotype was, in the opinion of the inventors, attributed to the fact that an increase in active gibberellin level was caused by the suppression of AtGA2ox4 gene expression and controlled in a feedback mechanism of GA 20-oxidse and GA 3-oxidase.

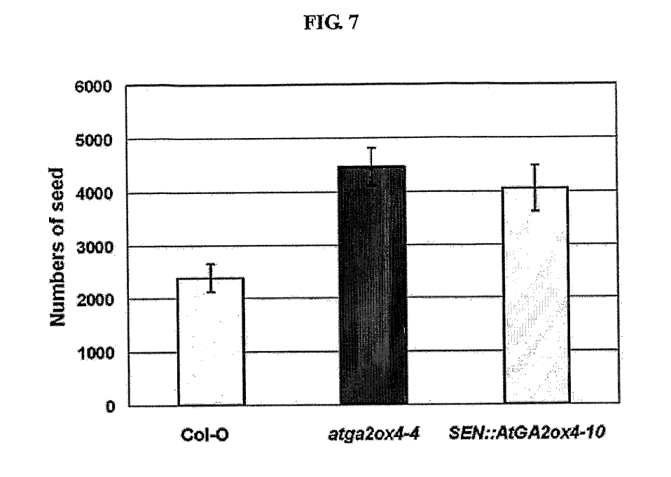

Interestingly, the mutant lines with dwarfism induced therein were found to be increased in seed productivity as compared to the wild-type. The SEN::AtGA2ox4-10 mutant line transformed with the pSEN-AtGA2ox4 construct, as shown in FIG. 7, produced a greater number of seeds than did Col-O (wild-type). This increased productivity indicated that the dwarfism induced through the overexpression of the AtGA2ox4 gene according to the present invention might be applied to other crops to increase crop yield. In FIG. 7, “the numbers of seeds” means numbers of seeds produced by one individual plant.

### Example 2-3

### Expression of Genes Responsible for GA 2-Oxidase and Flowering in SEN::AtGA2ox4 Mutant of Arabidopsis thaliana

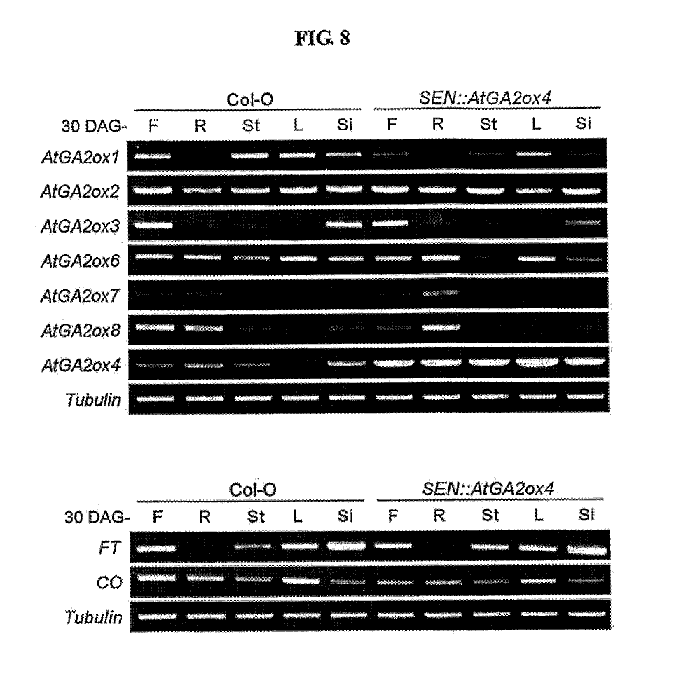

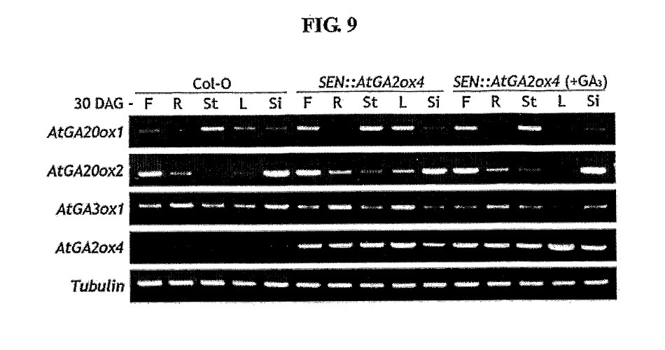

Genes associated with GA 2-oxidase activity, flowering and a feedback mechanism of the gibberellin metabolism in the SEN::AtGA2ox4 mutant with a dwarfism phenotype were analyzed for expression patterns. In this regard, total RNA was isolated from flowers, roots, stems, leaves and siliques of the wild-type Arabidopsis thaliana and the SEN::AtGA2ox4 mutant, both grown for 30 days after germination, with the aid of RNasey Plant Mini Kit (QIAGEN, Germany). cDNA was synthesized from 1 μg of each RNA using Superscript III Reverse Transcriptase (INVITROGEN, USA) under the conditions of 65° C., 5 min; 50° C., 60 min; and 70° C., 15 min. Then, PCR was performed using the synthesized cDNAs as templates in the presence of the primers, specific for GA 2-oxidase and flowering genes, listed in Table 1, below. The PCR was initiated by denaturing the template DNA at 94° C. for 2 min and performed with 30 cycles of 94° C., 1 min; 55° C., 1.5 min; and 72° C., 1 min, followed by extension at 72° C. for 15 min. The PCR products thus obtained were identified on 1% agarose gel by electrophoresis. The results are given in FIGS. 8 and 9.

As shown in FIG. 8, the AtGA2ox4 gene was expressed in flowers, roots, stems and siliques of the wild-type Arabidopsis thaliana grown for 30 days after germination, but almost not in the leaves. As for the expression levels of the gene, they were weaker in the stems than in the flowers, roots and siliques. On the basis of this observation, it was inferred that the action of the gene might be effected mainly in sink organs, such as flowers, roots and siliques, but almost not in the source organ of the normal plant, such as leaves. On the other hand, the SEN::AtGA2ox4 mutant showed increased expression levels of the gene in all organs, as compared to the wild-type. Particularly, the gene expression was greatly increased in leaves of the mutant, but almost no change was noticeable in those of the wild-type. This data indicates that the overexpression mechanism of AtGA2ox4 through the pSEN-AtGA2ox4 construct is effected mainly in leaves, inducing dwarfism.

The overexpression of AtGA2ox4 was found to have an influence on the expression patterns of GA 2-oxidase-related genes as follows. Among the enzymes using C₁₉-Gas (gibberellins) as substrates, AtGA2ox2 and AtGA2ox6, both of which are found in all organs, did not show a significant difference in expression level between the wild-type and the mutant. However, the expression level of AtGA2ox2 was slightly lowered in the leaves of the mutant. On the other hand, AtGA2ox1, which is almost not expressed in roots, was found to be decreased in expression level in the leaves and stems of the mutant as compared to the wild-type. As for AtGA2ox3 which is not found in leaves, its expression level was decreased in stems of the mutant. Turning to enzymes using C₂₀-Gas (gibberellins) as substrates, AtGA2ox7 was expressed specifically in flowers and roots and AtGA2ox8 was expressed at high levels in flowers and roots and at relatively low levels in siliques. Like AtGA2ox4, these genes were almost not expressed in the leaves. When compared to the wild-type, the mutant showed an increased expression level of AtGA2ox7 in roots. Likewise, the expression level of AtGA2ox8 was also increased in roots. Interestingly, the expression level of the genes in stems was lowered in the mutant. The overexpression of AtGA2ox7 and AtGA2ox8, which use C₂₀-Gas (gibberellins) as substrates, is known to induce dwarfism, like the AtGA2ox4 gene of the present invention, in Arabidopsis thaliana (Schomburg et al., 2003). This study data showed that the expression of AtGA2ox7 and AtGA2ox8, which use C₂₀-GAs (gibberellins) as substrates, induce various metabolisms in the sink organ root, leading to dwarfism while the overexpression of the AtGA2ox4 gene of the present invention, which uses C₁₉-Gas (gibberellins) as substrates, induces various metabolisms mainly in the source organ leaf, leading to dwarfism therein. It is suggested that the expression of AtGA2ox7 and AtGA2ox8 which use C₂₀-Gas (gibberellins) as substrates be regulated, directly or indirectly, by the expression of AtGA2ox4. This suggestion requires additional studies on whether plant dwarfism is induced by the gene of the present invention alone or in combination with AtGA2ox7 and AtGA2ox8.

An examination was made about whether the gene of the present invention is involved in the feedback mechanism of the gibberellin metabolism and thus in gibberellin catabolism. For this, AtGA20ox1 and AtGA20ox2, both coding for GA20-oxidase, and AtGA3ox1 coding for GA3-oxidase were analyzed for expression levels in the SEN::AtGA2ox4 mutant. As shown in FIG. 9, the SEN::AtGA2ox4 mutant was increased in expression levels for all of the genes, compared to the wild-type, particularly in the leaves. Also, treatment with GA3 was found to return the expression levels to those of the wild-type. On the basis of this observation, it can be inferred that the overexpression of AtGA2ox4 induces gibberellin insufficiency, leading to the induction of gibberellin synthesis genes. Therefore, the gene of the present invention is identified to play an important role in the catabolism of gibberellins. The gibberellin insufficiency attributed to the expression of the gene according to the present invention is believed to induce the expression of genes associated with gibberellin synthesis. Also, this gene expression regulation is inferred to be conducted predominantly in the leaves.

TABLE 1
SEQ ID NOS. of Primers

| | | SEQ ID NOS of Sense/Antisense |
|---|---|---|
| Nos. | Gene Names | Primers |
| 1 | AtGA2ox1 | SEQ ID NO. 7/SEQ ID NO. 8 |
| 2 | AtGA2ox2 | SEQ ID NO. 9/SEQ ID NO. 10 |
| 3 | AtGA2ox3 | SEQ ID NO. 11/SEQ ID NO. 12 |
| 4 | AtGA2ox4 | SEQ ID NO. 13/SEQ ID NO. 14 |
| 5 | AtGA2ox5 | SEQ ID NO. 15/SEQ ID NO. 16 |
| 6 | AtGA2ox6 | SEQ ID NO. 17/SEQ ID NO. 18 |
| 7 | AtGA2ox7 | SEQ ID NO. 19/SEQ ID NO. 20 |
| 8 | FT | SEQ ID NO. 21/SEQ ID NO. 22 |
| 9 | CO | SEQ ID NO. 23/SEQ ID NO. 24 |
| 10 | Tubulin (Positive | SEQ ID NO. 25/SEQ ID NO. 26 |
| | Control ) | |
| 11 | AtGA20ox1 | SEQ ID NO. 27/SEQ ID NO. 28 |
| 12 | AtGA20ox2 | SEQ ID NO. 29/SEQ ID NO. 30 |
| 13 | AtGA3ox1 | SEQ ID NO. 31/SEQ ID NO. 32 |

### Example 3

### Phenotype Recovery of SEN::AtGA2ox4 Mutant by Treatment with GA₃

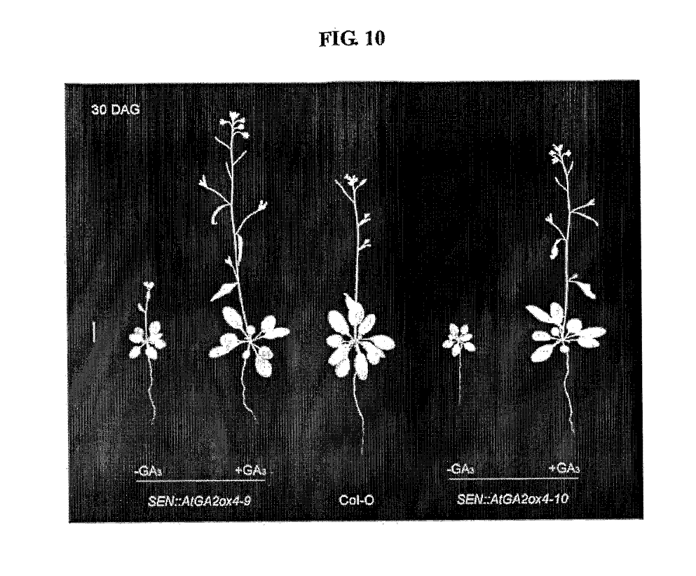

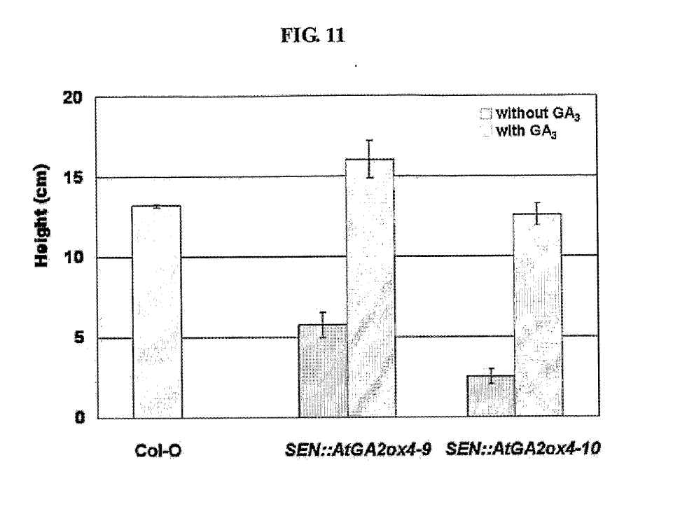

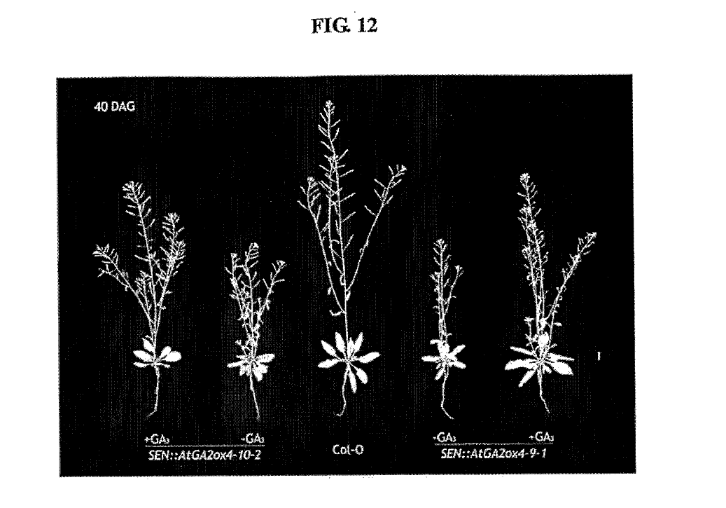

As suggested above, the AtAtGA2ox4 gene was inferred to have a GA 2-oxidase function which is involved in gibberellin catabolism. In order to examine whether the AtGA2ox4 gene is actually involved in gibberellin catabolism, the SEN::AtGA2ox4-9 and the SEN::AtGA2ox4-10 line, both showing dwarfism phenotypes, were grown for 30 days while 10⁻⁴ M GA₃ (Sigma, USA) was applied twice at intervals of one week starting from 12 days after germination. Treatment with the active gibberellin GA₃ may recover the phenotype of the dwarfism-induced mutant in which gibberellin insufficiency was caused by the overexpression of the AtGA2ox4 gene, to that of the wild-type. Comparisons were made between the mutants treated with or without GA₃ (FIGS. 10, 11 and 12). The mutant lines which were not treated with GA3 showed dwarfism to various extents depending on the expression levels of the gene. On the other hand, when treated with GA₃, the SEN::AtGA2ox4-9 line and the SEN::AtGA2ox4-10 line were recovered almost perfectly to the wild-type phenotype. Even the SEN::AtGA2ox4-10 line, which shows severe dwarfism, was found to be recovered to the phenotype of the wild-type when treated with GA₃. Meanwhile, the mutants which showed dwarfism phenotypes due to the overexpression of the gene were not different in flowering time from the wild-type. This was true of GA₃-treated mutants. These facts allow an inference that the gene of the present invention plays an important role in dwarfism induction in plants, but is not involved in the control of flowering time. Thus, the transgenic plant with a sense construct of the AtGA2ox4 gene was identified to be auxotrophic for GA₃, indicating that the polynucleotide encoded by the gene of the present invention may be a target for developing novel functional crops which do not require flowering time control, but need to be dwarfed.

### Example 4

### Analysis of Proteins of SEN::AtGA2ox4 Mutant

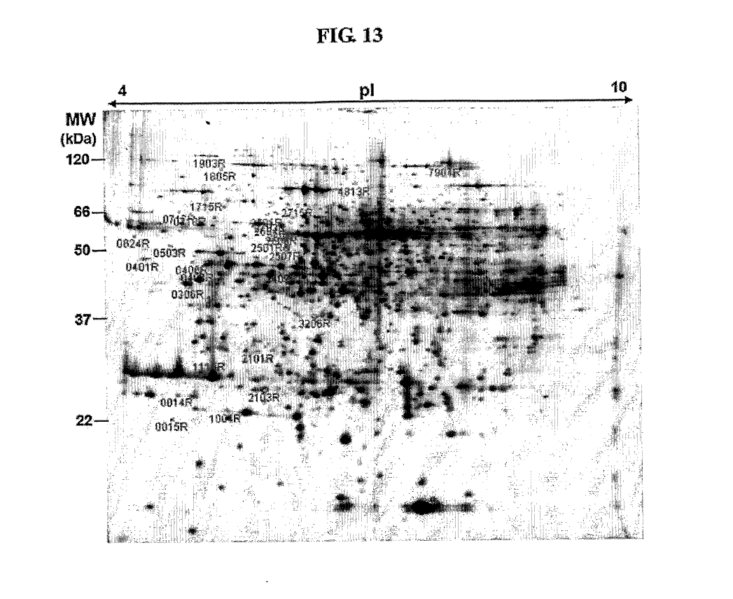

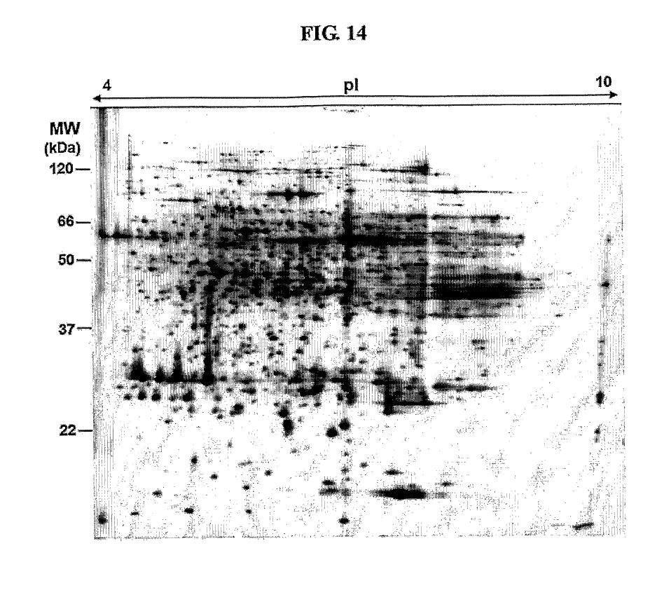

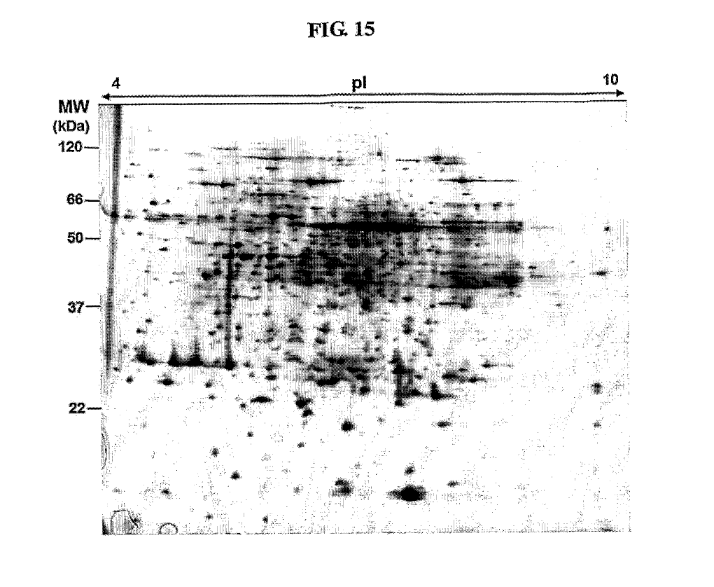

As described above, the overexpression of AtGA2ox4 was suggested to induce a dwarfism phenotype particularly in leaves. This suggestion was examined on a protein level. Proteins were isolated from the wild-type Arabidopsis thaliana (FIG. 13), the SEN::AtGA2ox4 mutant (FIG. 14), and the GA₃-treated SEN::AtGA2ox4 mutant (FIG. 15), all grown for 30 days after germination, and analyzed for expression pattern by two-dimensional electrophoresis. Protein isolation for each plant was conducted as follows. Each plant was mashed in 10 volumes of a reagent comprising 7M urea, 2M thiourea, 4% (w/v) 3-[(3-cholamidopropy)dimethyammonio]-1-propanesulfonate (CHAPS), 1% (w/v) dithiothreitol (DTT), 2% (v/v) pharmalyte and 1 mM benzamidine, followed by heating at 100° C. for 10 min. After centrifugation at 15,000 rpm for 1 hour, the supernatant was used as a sample for two-dimensional electrophoresis. Protein quantities were determined using the Bradford method (Bradford et al., 1976). For primary isoelectric focusing (IEF), IPG strips were soaked at room temperature for 12-16 hrs in a reswelling solution comprising 7M urea, 2M thiourea, 2% 3-[(3-cholamidopropy)dimethylammonio]-1-propanesulfonate (CHAPS), 1% dithiothreitol (DTT) and 1% pharmalyte. Each protein sample was used in the amount of 200 μg per strip. IEF was performed at 20° C. using a Multiphore II system from Amersham Biosciences according to the protocol provided by the manufacturer. For IEF, the voltage was linearly increased from 150 to 3500 V over 3 hours (to allow for sample entry), then the voltage was held constant at 3500 V with the focusing complete after 96 kVh. Prior to the second dimension SDS-PAGE, the strips were incubated for 10 min in equilibration buffer (50 mM Tris-Cl, pH6.8, 6M urea, 2% SDS, 30% glycerol) first with 1% DTT and second with 2.5% iodoacetamide. Each equilibrated strip was then put onto SDS-PAGE gel (20×24-cm 10˜16%), and the second dimension was run at 20° C. for 1.7 kVh using a Hoefer DALT 2D system (Amersham Biosciences). After the two-dimensional electrophoresis, the gel was silver-stained for visualization according to the Oakley method (Anal. Biochem. 1980, 105:361-363). The glutaraldehyde treatment was omitted for protein identification with a mass analyzer. The silver-stained, two-dimensional electrophoresis gel was scanned using an AGFA Duoscan T1200. The protein spots were quantified to examine a change in expression level by the analysis of the scanned images using the PDQuest software (version 7.0, BioRad). The quantity of each spot was normalized according to the total intensity of valid spots. Selected protein spots were digested with modified porcine trypsin according to the Shevchenko method (1996). The gel fragments were washed with 50% acetonitrile to remove impurities such as SDS, organic solvent, staining reagents, etc. Then, the gels were reswelled in trypsin (8˜10 ng/μl) and incubated at 37° C. for 8˜10 hours. This protein degradation was terminated with 5 μl of 0.5% trifluoroacetic acid. The trypsin digests of proteins were recovered as aqueous solutions which were desalted and concentrated to a volume of 1˜5 μl using C18ZipTips (Millipore). The concentrate was mixed with the same volume of a-cyano-4-hydroxycinnamic acid saturated with 50% aqueous acetonitrile and loaded on target plates for mass analysis with Ettan MALDI-TOF (Amersham Biosciences). The protein fragments loaded on the target plates were evaporated with an N₂ laser at 337 nm using a delayed extraction approach. They were accelerated with a 20-Kv injection pulse to analyze the time of flight. Each mass spectrum was the cumulative average of 300 laser shots. Spectra were calibrated with the trypsin autodigestion ion peaks m/z (842.510, 2211.1046) as internal standards. The search program ProFound, developed by Rockefeller University (http://129.85.19.192/profound_bin/WebProFound.exe), was used for protein identification using peptide mass fingerprinting.

Arabidopsis thaliana proteins which are up-regulated by the overexpression of the AtGA2ox4 gene and recovered to the phenotype of the wild-type by treatment with GA₃ are summarized in Table 2, below. Interestingly, almost no proteins which were down-regulated by the overexpression of the AtGA2ox4 gene were found. As seen in Table 2, more interestingly, the overexpression of the AtGA2ox4 gene induces an increase in the expression of a significant number of chloroplast target proteins. Over 50% of the proteins analyzed in the present invention were identified as chloroplast target proteins, and the other proteins were cytosol and other organelle target proteins. This arrangement was closely related with the specific expression of the AtGA2ox4 in leaves of the SEN::AtGA2ox4 mutant. Accordingly, the specific expression of the AtGA2ox4 gene in leaves leads to increasing the expression of various chloroplast target proteins related to dwarfism induction. In addition, gibberellin signaling-related proteins and endo- and exogenous environment related proteins were expressed on an elevated level mainly in the cytosol and other organelles. Taken together, this data indicates that the AtGA2ox4 gene induces the expression of chloroplast target proteins in the source organ leaf and leads to character appearance in the sink organs, resulting in dwarfism.

TABLE 2
Proteins up-regulated by AtGA2ox4 overexpression and recovered by GA₃ treatment

| | | | |
|---|---|---|---|
| Spot No. | Mw | Arabidopsis protein Name | Locus Tag. |

Chloroplast target proteins

| | | | |
|---|---|---|---|
| 14 | 25.92 | LHCB6 (LIGHT HARVESTING COMPLEX PSII); chlorophyll binding | |
| 15 | 22.30 | ATP-dependent Clp protease proteolytic subunit | |
| 306 | 43.09 | SBPASE (sedoheptulose-bisphosphatase); phosphoric ester hydrolase | AT3G55800 |
| 401 | 48.75 | RPS1 (ribosomal protein 51); RNA binding | AtSG30510 |
| 408 | 46.25 | CHLI1 (CHLORINA 42); magnesium chelatase | AT4G18480 |
| 624 | 54.07 | ribulose-1,5 bisphosphate carboxylase oxygenase large subunit N-methyltransferase, putative | |
| 1004 | 23.48 | LHCA4 (Photosystem I light harvesting complex gene 4); chlorophyll binding | |
| 1115 | 31.24 | chlorophyll a/b binding protein (LHCP AB 180) | |
| 1715 | 73.57 | ABC1 family protein | AT4G31390 |
| 2101 | 32.37 | CA1 (CARBONIC ANHYDRASE 1); carbonate dehydratase/zinc ion binding | |
| 2103* | 26.59 | ATFER1 (ferretin 1); ferric iron binding | AT5G01600 |
| 2409 | 45.98 | 3-isopropylmalate dehydrogenase, chloroplast, putative | AT5G14200 |
| 2507 | 50.73 | ADG1 (ADP GLUCOSE PYROPHOSPHORYLASE SMALL SUBUNIT 1); glucose-1-phosphate | AT5G48300 |
| | | adenylyltransferase | |
| 2604 | 59.62 | ATP synthase CF1 alpha subunit | |
| 2701 | 64.11 | ALDH10A8 (Aldehyde dehydrogenase 10A8); 3-chloroallyl aldehyde dehydrogenase | AT1G74920 |

Cytosol and other organelle target proteins

| | | | |
|---|---|---|---|
| 503** | 51.78 | 26s proteasome AAA•ATPase subunit RPT5a | AT3G05530 |
| 1805** | 96.01 | UBP14 (UBIQUITIN-SPECIFIC PROTEASE 14); ubiquitin-specific protease | AT3G20630 |
| 717*** | 66.33 | RCN1 (ROOTS CURL IN NPA): protein phosphatase type 2A regulator | AT1G25490 |
| 1903* | 123.37 | Transcription factor/transcriptional activator; response to stress | AT3G19290 |
| 2715* | 69.35 | putative 2,3-bisphosphoglycerate-independent phosphoglycerate mutase | AT1G09780 |
| 0406 | 48.04 | Serpin, putative/serine protease inhibitor, putative | AT1G47110 |
| 0718 | 64.88 | Protein phsphatase 2A 65 kDa regulatory subunit | |
| 2501 | 52.84 | Gamma-glutamylcystein synthetase | |
| 2606 | 54.92 | Strong similarity to alanine aminotransferase | AT1G17290 |
| 2703 | 61.15 | Dihydrolipoamide acetyltransferase | AT3G13930 |
| 7904 | 99.47 | Aconitase | AT3G16420 |

*indicates Stress-related proteins;
**indicates GA signalling-related proteins;
***indicates regulating proteins of hypocotyl elongation.

1.-20. (canceled)

21. A method of preparing a dwarfed plant, comprising:
(I) overexpressing a gene encoding an amino acid sequence of SEQ ID NO. 2; and
(II) selecting a dwarfism phenotype-induced plant.

22. The method according to claim 21, wherein the step (I) is carried out by transforming a plant with a gene encoding an amino acid sequence of SEQ ID NO 2.

23. The method according to claim 22, wherein the gene is a gene set forth in SEQ ID NO. 1.

24. The method according to claim 21, wherein the step (I) is carried out by transforming a plant with a recombinant vector carrying a gene encoding an amino acid sequence of SEQ ID NO. 2.

25. The method according to claim 21, wherein the step (I) is carried out by transfecting a plant with an Agrobacterium tumefaciens transformed with a recombinant vector which carries a gene encoding an amino acid sequence of SEQ ID NO. 2.

26. A dwarfed plant, prepared by the method of claim 21.

27. A method of preparing a plant having an improvement in seed productivity, comprising:
(I) overexpressing a gene encoding an amino acid sequence of SEQ ID NO. 2; and
(II) selecting a dwarfism phenotype-induced plant.

28. The method according to claim 27, wherein the step (I) is carried out by transforming a plant with a gene encoding an amino acid sequence of SEQ ID NO 2.

29. The method according to claim 28, wherein the gene is a gene set forth in SEQ ID NO. 1.

30. The method according to claim 27, wherein the step (I) is carried out by transforming a plant with a recombinant vector carrying a gene encoding an amino acid sequence of SEQ ID NO. 2.

31. The method according to claim 27, wherein the step (I) is carried out by transfecting a plant with an Agrobacterium tumefaciens transformed with a recombinant vector which carries a gene encoding an amino acid sequence of SEQ ID NO.

32. A plant having an improvement in seed productivity, prepared by one of the methods of claim 27.


SEQUENCE LISTING (32 sequences)

SEQ ID NO: 1 - DNA, 966 bp, Arabidopsis thaliana
atggtgaaagggtcccagaaaatcgtggccgtagatcaagacataccaataatagacatgtcgcaggagagatcacaagtgtcgatgcagatagtcaaagcctgcgagagtctcggcttcttcaaagtcatcaaccatggcgttgaccaaaccaccatctcaagaatggagcaagagtctataaacttctttgctaaaccggctcacgagaagaaatctgtccgaccagttaaccagcctttccggtatggttttagagacattggactcaacggtgactctggtgaggtcgagtatttgctgtttcacactaacgaccctgcctttcgctctcagctctccttcagctcggcagtgaattgttacatagaagcagttaagcagttggctcgtgagatcttagatctgacggctgagggacttcatgtcccacctcacagtttcagtaggttaatcagctccgtcgatagtgactccgttctgagagtgaatcattatccaccgtccgatcaattctttggtgaagccaatctttctgaccaatctgtgtcactgacaagagttggcttcggagaacacaccgaccctcagattttaacagttcttagatctaacggtgtaggagggctccaagtgtccaattcagatggcatgtgggtttctgtctcccctgacccttcagctttctgcgtcaatgtaggagacttgttacaggtgatgacgaacgggagatttataagtgtaaggcatagagcattgacctacggagaagaaagccggctatccacggcgtactttgccggaccaccgcttcaggcgaagattgggcctctttcggcgatggttatgacgatgaatcagccacggttgtaccaaacatttacttggggcgagtacaagaaacgtgcgtactctctacgacttgaggatagccgtttagacatgtttcgtacatgtaaggactag

SEQ ID NO: 2 - protein, 321 aa, Arabidopsis thaliana
MVKGSQKIVAVDQDIPIIDMSQERSQVSMQIVKACESLGFFKVINHGVDQTTISRMEQESINFFAKPAHEKKSVRPVNQPFRYGFRDIGLNGDSGEVEYLLFHTNDPAFRSQLSFSSAVNCYIEAVKQLAREILDLTAEGLHVPPHSFSRLISSVDSDSVLRVNHYPPSDQFFGEANLSDQSVSLTRVGFGEHTDPQILTVLRSNGVGGLQVSNSDGMWVSVSPDPSAFCVNVGDLLQVMTNGRFISVRHRALTYGEESRLSTAYFAGPPLQAKIGPLSAMVMTMNQPRLYQTFTWGEYKKRAYSLRLEDSRLDMFRTCKD

SEQ ID NO: 3 - DNA, 29 bp, Artificial Sequence
  Sense Primer
ggatccatggtgaaagggtcccagaaaat

SEQ ID NO: 4 - DNA, 31 bp, Artificial Sequence
  Anti-sense Primer
ggtgaccctagtccttacatgtacgaaacat

SEQ ID NO: 5 - DNA, 30 bp, Artificial Sequence
  Sense Primer
ggtgaccatggtgaaagggtcccagaaaat

SEQ ID NO: 6 - DNA, 30 bp, Artificial Sequence
  Anti-sense primer
ggatccctagtccttacatgtacgaaacat

SEQ ID NO: 7 - DNA, 25 bp, Artificial Sequence
  Sense Primer
caataagaaaaccaatggtggtaag

SEQ ID NO: 8 - DNA, 25 bp, Artificial Sequence
  Anti-sense Primer
tacgaaacaaacaaacacactcttg

SEQ ID NO: 9 - DNA, 25 bp, Artificial Sequence
  Sense Primer
gattaaggaagtgtcgtacaaggtg

SEQ ID NO: 10 - DNA, 25 bp, Artificial Sequence
  Anti-sense Primer
ccatttccttctttttgtgattatg

SEQ ID NO: 11 - DNA, 25 bp, Artificial Sequence
  Sense Primer
taaagagatgaagagaatgtcgagc

SEQ ID NO: 12 - DNA, 25 bp, Artificial Sequence
  Anti-sense Primer
caaacattggatagagaaaatggag

SEQ ID NO: 13 - DNA, 25 bp, Artificial Sequence
  Sense Primer
tcgagtatttgctgtttcacactaa

SEQ ID NO: 14 - DNA, 25 bp, Artificial Sequence
  Anti-sense Primer
gtacgaaacatgtctaaacggctat

SEQ ID NO: 15 - DNA, 27 bp, Artificial Sequence
  Sense Primer
atatctcatgatgatcctttcaagttc

SEQ ID NO: 16 - DNA, 27 bp, Artificial Sequence
  Anti-sense Primer
ggacatctaaacggagagagtatgtag

SEQ ID NO: 17 - DNA, 25 bp, Artificial Sequence
  Sense Primer
tttcacatcattctttcagaggttt

SEQ ID NO: 18 - DNA, 25 bp, Artificial Sequence
  Anti-sense Primer
aagcctaccttatcaccagtttctt

SEQ ID NO: 19 - DNA, 27 bp, Artificial Sequence
  Sense Primer
gacaacaaggactttactactctcagc

SEQ ID NO: 20 - DNA, 27 bp, Artificial Sequence
  Anti-sense Primer
aaaccaaacttcttaacatcttcttga

SEQ ID NO: 21 - DNA, 18 bp, Artificial Sequence
  Sense Primer
acgccatcagcgagttcc

SEQ ID NO: 22 - DNA, 24 bp, Artificial Sequence
  Anti-sense Primer
aaatgtatgcgttatggttaatgg

SEQ ID NO: 23 - DNA, 25 bp, Artificial Sequence
  Sense Primer
acaactggaacaacctttggcaatg

SEQ ID NO: 24 - DNA, 30 bp, Artificial Sequence
  Anti-sense Primer
actataggcatcatcaccgttcgttactcg

SEQ ID NO: 25 - DNA, 21 bp, Artificial Sequence
  Sense Primer
ctcaagaggttctcagcagta

SEQ ID NO: 26 - DNA, 21 bp, Artificial Sequence
  Anti-sense Primer
tcaccttcttcatccgcagtt

SEQ ID NO: 27 - DNA, 19 bp, Artificial Sequence
  Forward primer
cctctcatcgaccttaacc

SEQ ID NO: 28 - DNA, 23 bp, Artificial Sequence
  Reward primer
tcctagtgtgagatctggtttac

SEQ ID NO: 29 - DNA, 20 bp, Artificial Sequence
  Forward primver
gtcactaatagcggatgctc

SEQ ID NO: 30 - DNA, 20 bp, Artificial Sequence
  Reward primer
ggaatcgagagtattcacat

SEQ ID NO: 31 - DNA, 37 bp, Artificial Sequence
  Forward primer
gccggatccaattaaaaaagagcaagatgcctgstat

SEQ ID NO: 32 - DNA, 36 bp, Artificial Sequence
  Reward primer
aaaggtacctgttcctcgtactcttcaacgatatcg